(19) United States
(12) Patent Application Publication
(10) Pub. No.: US 2008/0236574 A1
(43) Pub. Date: Oct. 2, 2008

(54) MOUTHPIECE FOR PREVENTING FORMATION OF PERIORAL RHYTIDS

(76) Inventor: Ann Marx, Wayne, IL (US)

Correspondence Address: FOX ROTHSCHILD LLP;PRINCETON PIKE CORPORATE CENTER, 2000 Market Street, Tenth Floor, Philadelphia, PA, 19103, US

(21) Appl. No.: 11/695,243

(22) Filed: Apr. 2, 2007

(51) Int. Cl. A61M 11/00

(52) U.S. Cl. 128/200.23

(57) ABSTRACT

A mouthpiece for preventing perioral rhytids is provided. The mouthpiece comprises an inlet end adapted to receive fluid from a container and an outlet end spaced from the inlet end and in fluid communication with the inlet end, wherein the outlet end is sized to reduce the contraction of the orbicularis oris muscle of a user while receiving fluid from the container. The inlet end of the mouthpiece may be designed to be removably attached to the container directly or remotely. In some embodiments, the mouthpiece may further include a seal and a flow control plate. A method for preventing perioral rhytids using such mouthpiece is also disclosed.

### FIELD OF THE INVENTION

This invention relates to a mouthpiece for delivering fluids to the user's mouth from a container. More particularly, it discloses a mouthpiece for preventing formation of perioral rhytids.

### BACKGROUND OF THE INVENTION

Perioral rhytids (wrinkles around the mouth) have many etiologies. Sometimes called “smokers lines”, we know this is not the only cause. Any action or movement which causes contraction of the Orbicularis oris (circular muscle around the mouth used for speaking, eating, drinking, whistling, kissing, etc.) will over time cause vertical wrinkles to form around the mouth. The cumulative result of these actions is the permanent formation of wrinkles around the mouth even in the absence of any movement in this area.

One common etiology in these modern times causing these wrinkles, is the widespread use of small necked bottles for the ingestion of water and other drinks. While fluid intake is beneficial, the use of small neck bottles necessarily cause contraction of the orbicularis oris muscle which over time results in these permanent vertical lines around the mouth.

The presence of these unsightly lines sends numerous women to their plastic surgeons office each year looking for ways to remove or diminish these unattractive features which are associated with a premature aged look. A substantial amount of monies are spent on various treatments in an attempt to eradicate these lines, the results of which are less than optimal.

Accordingly, there is a need for devices and methods that can decrease the amount of daily contractions of orbicularis oris muscle, thereby decreasing the occurrence of the perioral rhytids, delaying or possibly eliminating the need for treatment.

### SUMMARY OF THE INVENTION

In one aspect, a mouthpiece for delivering fluid to the user's mouth from a container is provided. The mouthpiece comprises an inlet end adapted to receive fluid form the container and an outlet end spaced from the inlet end and in fluid communication with the inlet end, wherein the outlet end is sized to reduce the contraction of the orbicularis oris muscle of a user while receiving fluid from the container. The inlet end of the mouthpiece is further adapted to be removably attached to the container. The mouthpiece may be attached to the container directly or remotely by using a hose.

The outlet end of the mouthpiece may be between about 5 mm and 15 mm wide and between about 25 mm and 55 mm long. The mouthpiece may also include a flow control plate or a seal.

In another aspect, a method for preventing perioral rhytids is provided. The method comprises attaching a mouthpiece as described above to a fluid container and causing fluid to be dispensed from the container to a user's mouth.

### BRIEF DESCRIPTION OF THE DRAWINGS

For the purpose of illustrating the invention, there is shown in the drawings a form that is presently preferred; it being understood, however, that this invention is not limited to the precise arrangements and instrumentalities shown.

FIG. 1 is a front view of a mouthpiece attached to a fluid container.

FIGS. 2a-2d illustrate different suitable shapes for a middle section of the mouthpiece shown in FIG. 1.

FIG. 3a and FIG. 3b illustrate top views of the mouthpiece shown in FIG. 1, illustrating different flow control plates.

FIG. 4a and FIG. 4b illustrate different types of seals present in some embodiments of the mouthpiece shown in FIG. 1.

### DETAILED DESCRIPTION

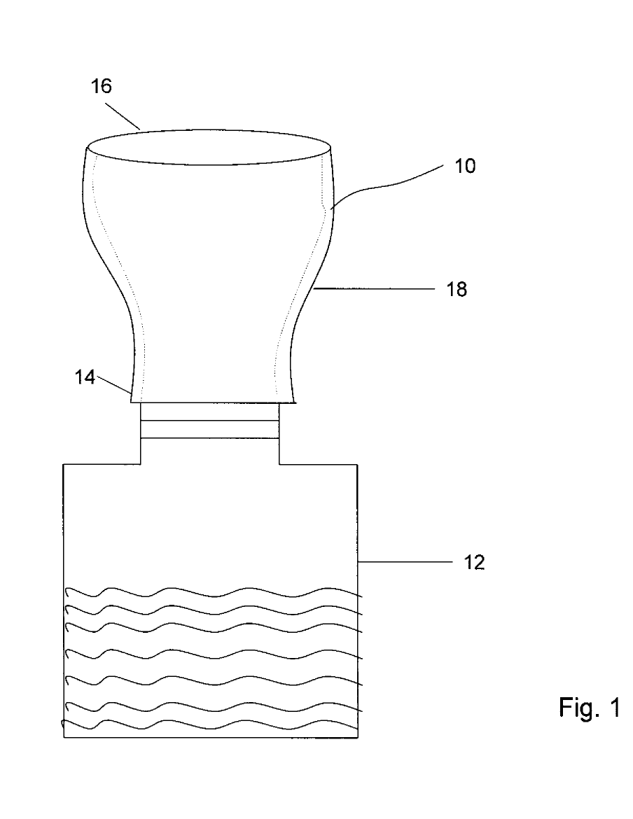

FIG. 1 illustrates a mouthpiece 10 attached to a fluid container 12. The container may be any type of container for storing fluids for human consumption. The mouthpiece 10 is mostly hollow and may preferably be made of any resilient flexible material such as, for example, rubber or plastic, although metals may also be used. Furthermore, preferably the selected material is inert and is easy to clean or sterilize, as necessary. In some embodiment, additives may be included to ensure that the mouthpiece has a pleasing feel or taste to the user. The mouthpiece 10 may comprise an inlet end 14 and an outlet end 16 in fluid communication with the inlet end 14.

The inlet end 14 is adapted to receive fluid from the container. In some embodiments, the mouthpiece may be adapted to be removably attached to the container. The mouthpiece may be attached directly to the container by any method known in the art such as screwing the mouthpiece onto the container, snapping the mouthpiece onto the container, or simply forcing the mouthpiece over the container outlet. Representative removable attachments are illustrated. For example in U.S. Pat. Nos. 4,230,229; 5,542,670; 6,230,923, and 6,386,395, incorporated herein by reference. In some embodiment, the mouthpiece may be attached to a remote container such as by attaching one end of a hose to a container and inserting the inlet end of the mouthpiece into the other end of the hose. Alternatively, the mouthpiece may be attached to a straw that can be placed into the container.

A person with ordinary skill in the art will undoubtedly be capable to design the mouthpiece 10 to fit any type of container conventionally used for holding fluids. By way of non-limiting example, it can be designed for small necked bottles such as Coca-Cola® bottles or large necked bottles such as quart-size Gatorade® bottles. In other embodiment, the mouthpiece may be designed to be attached to a soda can.

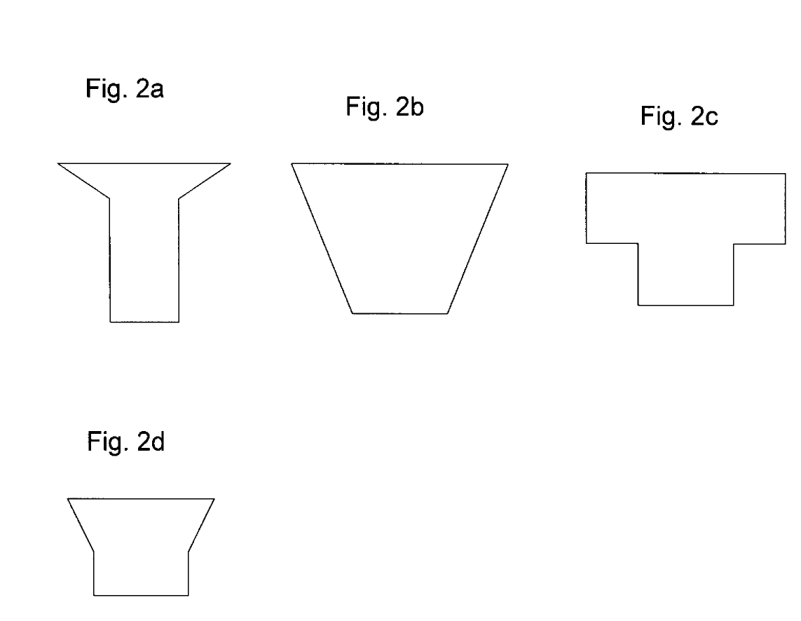

The outlet piece 16 is in fluid communication with the inlet end 14 to allow the user of the mouthpiece 10 to receive fluid from the container 12. The outlet end 16 may be separated from the inlet 14 by a middle section 18. The middle section 18 may be made of any practical dimensions or shape. Suitable non-limiting examples of mouthpieces with different middle sections are presented in FIGS. 2a, 2b, 2c and 2d.

The outlet piece 16 is sized to significantly eliminate the contraction of the user's orbicularis oris muscle while ingesting fluid from the container. The orbicularis oris muscle is the sphincter muscle responsible for maintaining constriction of the mouth. It closes the lips and is used for speaking, eating and drinking. Accordingly, in order to minimize the contraction of this muscle, the outlet end is preferably shaped to follow the natural shape of the human mouth, i.e. elongated ellipse among others. In the preferred embodiment, the outlet end is about 25 mm to 55 mm long and about 5 mm to 15 mm wide. The outlet end of the mouthpiece may be slightly curved in or concave to make it more comfortable for the user.

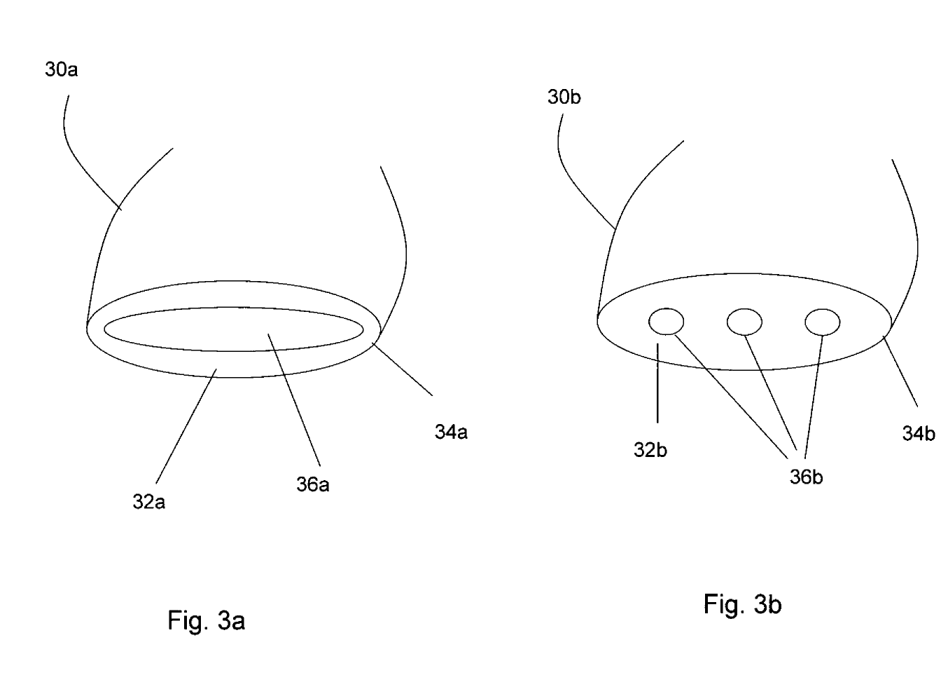

Referring to FIGS. 3a and 3b, the mouthpieces 30a and 30b may further include a flow control plate 32a and 32b respectively for controlling the flow of fluid through the mouthpiece. The flow control plate is preferably disposed at the outlet ends 34a and 34b as shown in FIG. 3a and FIG. 3b. Alternatively, it can be placed inside the mouthpiece or at the inlet end. The flow control plate includes openings 36a, 36b for dispensing fluid from the mouthpiece 32a, 32b. These openings may vary in number or shapes.

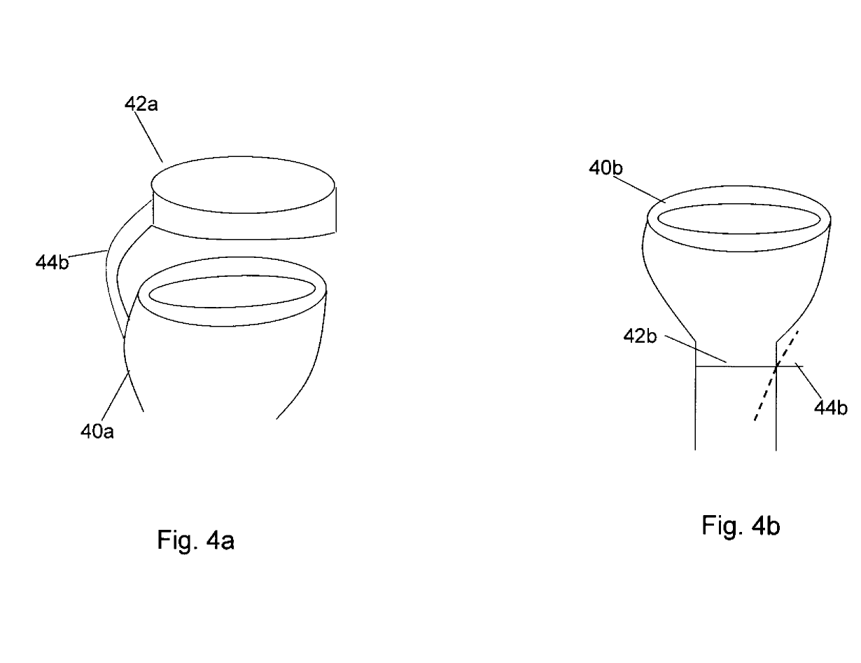

The mouthpiece may further comprise a seal. In some embodiments, as shown in FIG. 4a, the seal may be an external cover 42 that can be attached to the mouthpiece 40 to seal it. Preferably, the cover 42 may be connected to the mouthpiece 40 by a strap 44 to ensure that the cover is not lost. Such covers are known and are disclosed, for example, in U.S. Pat. Nos. 5,785,193 and 5,435,456, incorporated herein by reference.

Alternatively, the mouthpiece may comprise an internal seal. In some embodiments, as shown in FIG. 4b, the internal seal 42b inside the mouthpiece 40b may be moved from a closed position (represented by solid line) to an open position (represented by a dotted line) by simply moving it out of the way using an external lever 44b such as by flipping it up or down. In other embodiments, such internal seal may comprise a bite valve. In closed positions, the valve prevents fluid from spilling from the container. Biting on the valve compresses the valve and lets the fluid pass through the valve. Examples of bite valves are described, for example, in U.S. Pat. Nos. 5,601,207 and 6,364,168, incorporated herein by reference. Finally, the seal may comprise a push to close/pull to open type valve. Suitable non-limiting examples are described in U.S. Pat. No. 6,622,988, incorporated herein by reference.

A method for treatment of perioral rhytids is also provided. The method comprises attaching a mouthpiece as descried above to a fluid container and causing the fluid to be dispensed from the container to a user's mouth.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the following claims.

What is claimed is:

1. A mouthpiece for receiving fluid from a container comprising:
an inlet end adapted to receive fluid from the container; and
an outlet end spaced from the inlet end and in fluid communication with the inlet end, wherein the outlet end is sized to reduce the contraction of the orbicularis oris muscle of a user while receiving fluid from the container.

2. The mouthpiece of claim 1, wherein the inlet end is further adapted to be removably attached to the container.

3. The mouthpiece of claim 1, wherein the outlet end is between about 5 mm and 15 mm wide and between about 25 mm and 55 mm long.

4. The mouthpiece of claim 1 further comprising a flow control plate.

5. The mouthpiece of claim 1 further comprising a seal.

6. The mouthpiece of claim 1 wherein the mouthpiece is attached to the container directly.

7. The mouthpiece of claim 1 wherein the mouthpiece is attached to the container using a hose.

8. A method for preventing perioral rhytids comprising:
attaching a mouthpiece to a fluid container, wherein the mouthpiece comprises:
an inlet end adapted to receive fluid from the container;
an outlet end spaced from the inlet end and in fluid communication with the inlet end, wherein the outlet end is sized to reduce the contraction of the orbicularis oris muscle of a user while receiving fluid from the container; and
causing fluid to be dispensed from the container to a user's mouth.

9. The method of claim 8, wherein the inlet end is further adapted to be removably attached to the container.

10. The method of claim 8, wherein the outlet end is between about 5 mm and 15 mm wide and between about 25 mm and 55 mm long.

11. The method of claim 8, wherein the mouthpiece further comprises a flow control plate.

12. The method of claim 8, wherein the mouthpiece further comprises a seal.

13. The method of claim 8, wherein the mouthpiece is attached to the container directly.

14. The method of claim 8, wherein the mouthpiece is attached to the container using a hose.
